(19) United States
(12) Patent Application Publication
(10) Pub. No.: US 2012/0029411 A1
(43) Pub. Date: Feb. 2, 2012

(54) CARPAL TUNNEL DRAINAGE

(75) Inventor: Elaine Lee, Santa Clara, CA (US)

(73) Assignee: KYPHON SÀRL, Neuchatel (CH)

(21) Appl. No.: 12/845,668

(22) Filed: Jul. 28, 2010

(51) Int. Cl. A61M 5/00

(52) U.S. Cl. 604/8

(57) ABSTRACT

A procedure for treating carpal tunnel syndrome can involve aspirating one or more of the flexor tendons running through the carpal tunnel to reduce swelling, and hence reduce the cross-sectional area of those flexor tendons. As a result, the median nerve also running through the carpal tunnel is less constricted, and the CTS symptoms associated with such constriction can be relieved. Such relief can be enhanced by delivery of one or more therapeutic substances to the area to provide additional or supplemental symptom treatment.

### FIELD OF THE INVENTION

The invention relates to a system and method for minimally invasive surgical treatment of carpal tunnel syndrome.

### BACKGROUND OF THE INVENTION

The carpal tunnel is an opening through the wrist into the hand that is formed by the carpal bones of the wrist on the bottom and the transverse carpal ligament on the top. The transverse carpal ligament is at the base of the wrist and crosses from one side of the wrist to the other. It is sometimes referred to as the carpal ligament or the flexor retinaculum.

The median nerve and flexor tendons of the hand run through the carpal tunnel. The median nerve rests on top of the flexor tendons, just below the carpal ligament. It gives sensation to the thumb, index finger, long finger, and half of the ring finger. It also sends a nerve branch to control the muscles of the thumb.

In general, carpal tunnel syndrome (CTS) develops when the tissues around the median nerve swell and press on the nerve. Early in the disorder, the process is reversible. Over time, however, the insulation on the nerves may wear away, and permanent nerve damage and severe loss of hand function may develop, along with pain, numbness, and tingling in the wrist, hand, and fingers. Only the little finger is unaffected by the median nerve.

Virtually all workers who use their hands and wrists repetitively are at risk for CTS, particularly if they work in cold temperatures and have factors or medical conditions that make them susceptible. For example, computer users/typists, workers in the meat and fish packing industries, airplane assemblers, and musicians are among those at very high risk for CTS. In addition, people who intensively cook, knit, sew, do needlepoint, play computer games, do carpentry, or extensively use power tools are likewise at increased risk for CTS.

Ideally, the early phases of carpal tunnel syndrome are treated before the damage progresses. A conservative approach to CTS, which may include corticosteroid injections and splinting, is typically the first step in treating this disorder. The conservative approach is most successful in patients with mild carpal tunnel syndrome. A concurrent regimen of physical therapy (e.g., a program of hand/wrist stretching and strengthening) may provide further benefits. In addition, alternative therapies such as ultrasound, nonsteroidal anti-inflammatory drugs (NSAIDs), ice/warmth, low-level laser therapy (LLLT), dietary modification, and acupuncture have been used in the treatment of CTS, with varying degrees of success.

For severe cases of CTS, surgery may be a more effective treatment option. Surgery is also more likely to be necessary for patients with underlying conditions such as diabetes. Even among patients with mild CTS, there is a high risk of relapse. Some researchers are reporting better results when specific exercises for carpal tunnel syndrome are added to the program of treatments.

Traditionally, in a CTS surgery, the carpal ligament is cut free (“released”) from the median nerve, thereby relieving the pressure on the median nerve. The most common approach has been an open surgical procedure (“open carpal tunnel release” or “open release”) performed in an outpatient facility, and is a straightforward and well-characterized procedure.

In recent years, more surgeons have adopted a “mini” open—also called short-incision—procedure. This surgery requires only a one-inch incision, but it still allows a direct view of the area (unlike endoscopy, which is viewed on a monitor). The mini-open approach may allow for quicker recovery while avoiding some of the complications of endoscopy, although few studies have investigated its benefits and risks. The recovery time in patients receiving the mini-open approach may be shorter than with the open approach, and results are generally the same.

Endoscopy for CTS is another less invasive procedure than standard open release. In an endoscopic release procedure, a surgeon makes one or two ½-inch incisions in the wrist and palm, and inserts one or two endoscopes (pencil-thin tubes). The surgeon then inserts a tiny camera and a knife through the lighted tubes. While observing the underside of the carpal ligament on a screen, the surgeon cuts the ligament to free the compressed median nerve.

Endoscopic release patients report less pain than those who had the open release procedure, and return to normal activities in about half the time. Nevertheless, at this time the best evidence available does not show any significant long-term advantages of endoscopy over open release in terms of muscle, grip strength, or dexterity. The endoscopic approach may even carry a slightly higher risk of pain afterward. This may be due to a more limited view of the hand with endoscopy. Concerns of irreversible nerve injury with endoscopic carpal tunnel release, when compared with open carpal tunnel release, exist because of this reduced visibility.

Regardless of the procedure, patients who have undergone conventional CTS surgery typically report some permanent loss of grip strength, a loss of lifting strength in the wrist/forearm and nagging loss of full range of motion of the hand and wrist after surgery. This is due to the severing of the carpal ligament. The purpose of the carpal ligament is to wrap around the hand and wrist and hold the many small bones of the hand and wrist securely together. The carpal ligament also maintains the flexor tendons in a path that enables the mechanical advantage necessary for strong grip strength and range of motion. These support capabilities are naturally diminished when the ligament is severed.

Accordingly, it is desirable to provide a system and technique for alleviating the symptoms of CTS while maintaining as much grip strength and/or range of motion of the hand and wrist.

### SUMMARY OF THE INVENTION

By aspirating the tendons adjacent to the median nerve in the carpal tunnel, compression of that median nerve can be relieved, thereby reducing the symptoms of carpal tunnel syndrome (CTS). Such relief can be enhanced by delivery of one or more therapeutic substances to the area to provide additional or supplemental symptom treatment.

A procedure for treating CTS can involve aspirating one or more of the flexor tendons running through the carpal tunnel to reduce swelling, and hence reduce the cross-sectional area of those flexor tendons. As a result, the median nerve also running through the carpal tunnel is less constricted, and the CTS symptoms associated with such constriction can be relieved. In various embodiments, one or more drainage structures (e.g., needles or shunts) can be placed at selected flexor tendons, via either a previously created incision in the skin or through direct piercing of the skin. During this placement, each drainage structure can also penetrate the sheath of the target tendon. Once placed at or near the tendon(s), excess fluid can be aspirated through the drainage structure(s) to reduce swelling of the tendon(s). A flow controller such as a syringe, pump, or valve can be used to control the passage of aspirated material through each drainage structure.

In various other embodiments, the flexor tendon aspiration can be combined with localized therapeutic substance delivery to the carpal tunnel region to augment, enhance, and/or broaden the range of CTS symptom relief. For example, anti-inflammatory drugs, antibiotics, corticosteroids, pain medications, analgesics, anesthetics, relaxants, enzymes, lubricants, anti-fibrotic agents, desiccants, growth factors, and/or other therapeutic agents could be delivered to the flexor tendons, the transverse carpal ligament, and/or anywhere else in the general vicinity of the carpal tunnel. In some embodiments, the therapeutic substance delivery can be through the same drainage structure(s) (e.g., the same needle(s)) used for aspiration of the flexor tendons. In other embodiments, dedicated delivery structures (e.g., needles) can be used for the therapeutic substance delivery.

A surgical kit for treating CTS can include one or more drainage structures (e.g., needles or shunts) and instructions for using those drainage structures for aspirating one or more flexor tendons in the region of the carpal tunnel. In some embodiments, the instructions can describe the use of a single drainage structure for aspirating multiple tendons, and in other embodiments, the instructions can describe the use of multiple drainage structures.

In various other embodiments, the kit can further include a source and instructions for delivering a therapeutic substance from the source to the carpal tunnel region via the drainage structure(s) used for aspiration. In various other embodiments, the kit can further include a source, one or more delivery structures (e.g., needles), and instructions for delivering a therapeutic substance from the source via the one or more delivery structures to the vicinity of the carpal tunnel.

As will be realized by those of skilled in the art, many different embodiments of method for treating CTS using local tendon aspiration and optional therapeutic substance delivery, and/or a surgical kit for tendon aspiration are possible. Additional uses, advantages, and features of the invention are set forth in the illustrative embodiments discussed in the detailed description herein and will become more apparent to those skilled in the art upon examination of the following.

### BRIEF DESCRIPTION OF THE DRAWINGS

FIGS. 1A-1B show an example of median nerve compression due to tendon swelling in the carpal tunnel.

FIGS. 1C-1G show exemplary tendon aspiration treatments for the median nerve compression in the carpal tunnel.

FIG. 1H shows an exemplary result of tendon aspiration within the carpal tunnel.

FIG. 2 shows a kit including one or more drainage structures for tendon aspiration.

### DETAILED DESCRIPTION

By aspirating the tendons adjacent to the median nerve in the carpal tunnel, compression of that median nerve can be relieved, thereby reducing the symptoms of carpal tunnel syndrome (CTS). Such relief can be enhanced by delivery of one or more therapeutic substances to the area to provide additional or supplemental symptom treatment. Because needles can be used for both aspiration and therapeutic substance delivery, the procedure can be performed in a minimally invasive manner while still providing immediate, and potentially long-term, relief from the symptoms of CTS.

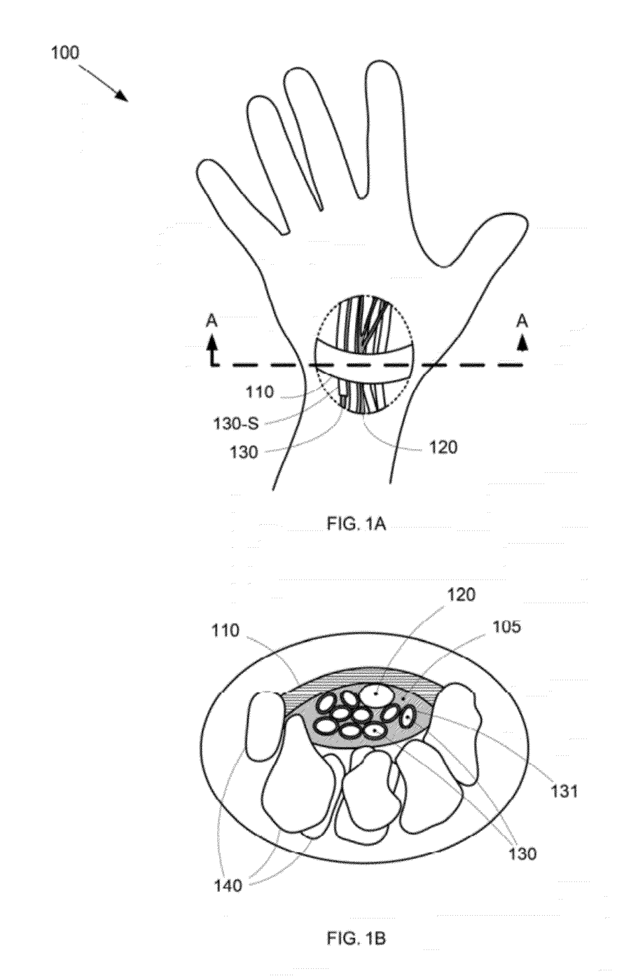

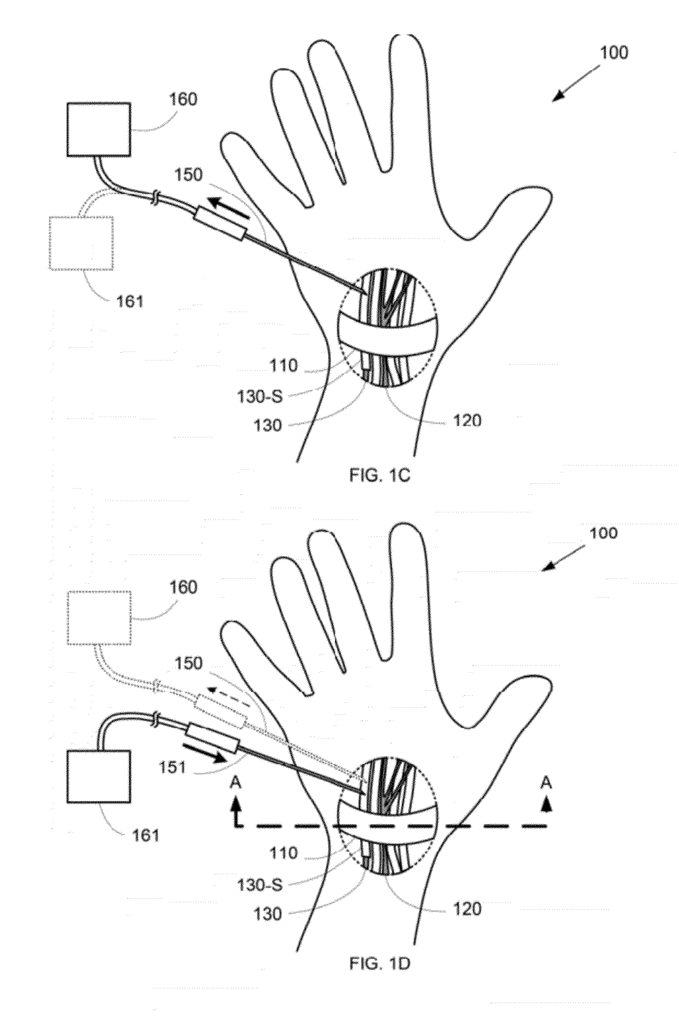

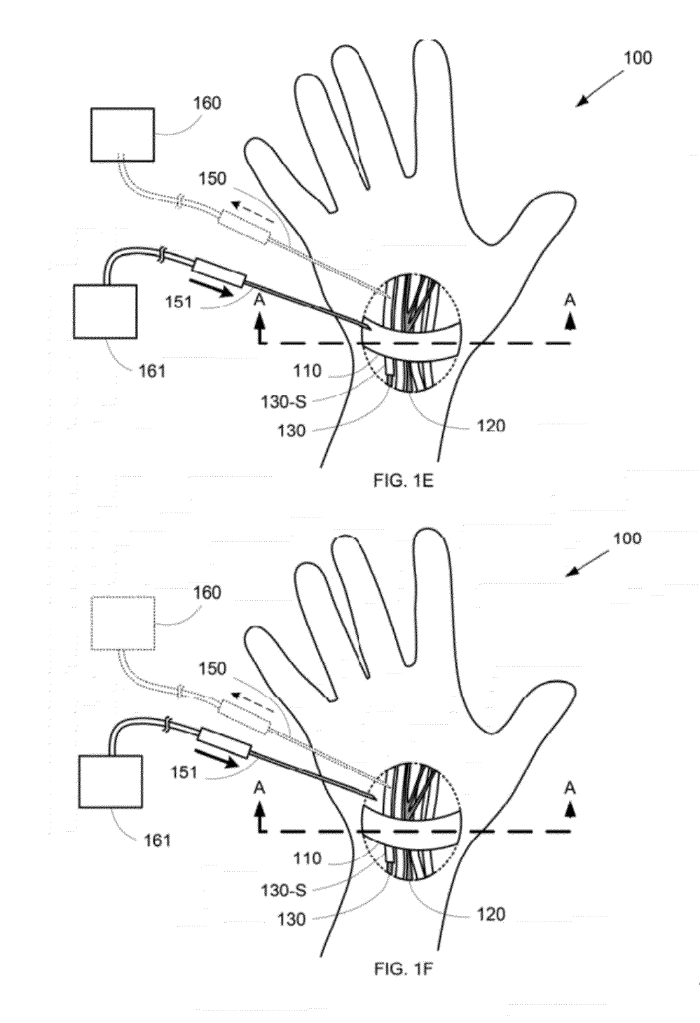

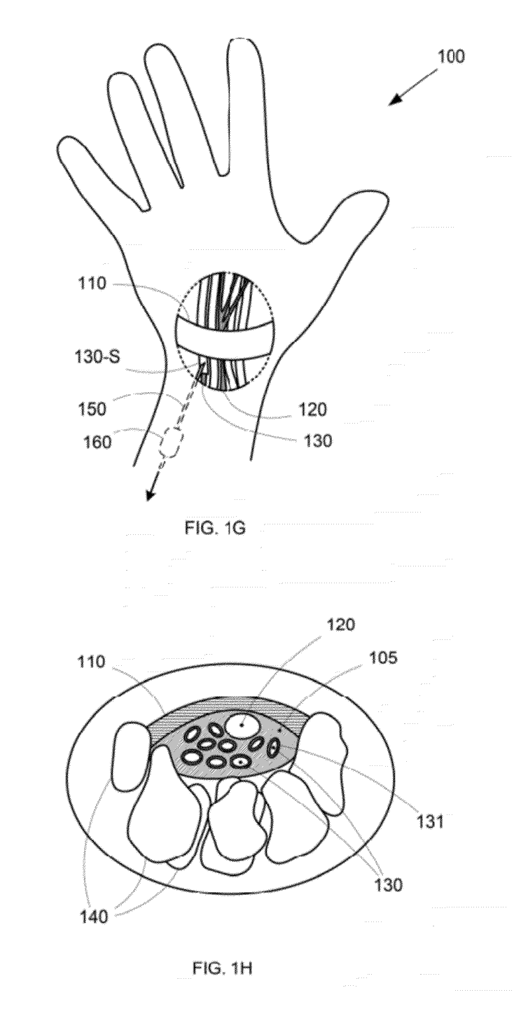

FIGS. 1A-1H show an exemplary procedure involving the aspiration of one or more tendons in the carpal tunnel for the treatment of CTS. In FIG. 1A, a cutaway view is shown of a hand 100 that shows the position of the median nerve 120 running beneath the transverse carpal ligament 110, along with the surrounding flexor tendons 130 that bend the fingers and thumb of hand 100. Each tendon 130 is covered by a sheath 130-S.

Note that hand 100 would typically be the hand of a patient suffering from CTS, with the below-described procedure being an exemplary treatment of that patient. However, in various other embodiments, hand 100 could be the hand of a cadaver, or simply a model hand, with the below-described procedure being performed for training/instructional purposes.

Sectional view A-A shown in FIG. 1B depicts the carpal tunnel 105 formed by the carpal ligament 110 and the wrist (carpal) bones 140. As noted above, each tendon 130 is encased within a sheath 130-S to facilitate movement. Impingement of median nerve 120 between carpal ligament 110 and flexor tendons 130 as shown in FIG. 4B causes CTS and its concomitant symptoms.

This problematic pressure on median nerve 120 can be due in large part to the swelling of flexor tendons 130 beyond their normal states. As indicated in FIG. 1B, flexor tendons 130 and median nerve 120 are all packed into the relatively narrow carpal tunnel 105. Therefore, any swelling of flexor tendons 130 will likely result in squeezing of median nerve 120. Oral diuretics or anti-inflammatory medications may alleviate this swelling somewhat, but are unlikely to have any significant local effect.

In contrast, local aspiration of flexor tendons 130 can provide immediate and significant reduction of swelling (and hence reduce compression of median nerve 120). In one embodiment, a drainage structure 150 (e.g., a needle or shunt) is placed at a selected tendon 130, as shown in FIG. 1C, optionally, puncturing sheath 130-S to reach ligament 130. A flow controller 160 can then be used to aspirate fluid from tendon 130, thereby reducing the swelling. Some or all of tendons 130 can be aspirated in this manner to provide more space in carpal tunnel 105 for median nerve 120.

Note that aspiration of a given flexor tendon 130 can result in a decrease in swelling along a portion of that tendon 130. In many instances, the specific location at which aspiration of the individual flexor tendons 130 takes place is not critical, so long as that location is substantially adjacent to carpal tunnel 105 (i.e., anywhere from the forearm through the palm). Accordingly, aspiration from anywhere within that range can often provide the desired thinning of tendon(s) 130 within carpal tunnel 105.

In various embodiments, flow controller 160 can be a syringe, vacuum pump, valve, or any other device for controlling the aspiration of material through drainage structure 150. Note that while vacuum source 160 is depicted as being remotely coupled to drainage structure 150 (e.g., via flexible tubing) for exemplary purposes, in various other embodiments, flow controller 160 could be rigidly coupled to drainage structure 150 (e.g., a syringe fitted with drainage structure 150).

A single drainage structure 150 is depicted for exemplary purposes, and that same drainage structure 150 could be used to aspirate any number of flexor tendons 130. However, in various other embodiments, multiple drainage structures 150 can be used to aspirate flexor tendons 130. Such aspiration could be performed concurrently or sequentially to remove fluid causing swelling of tendons 130.

In some embodiments, the aspirated fluid may be embedded or integrated in a carrier structure or matrix (e.g., a desiccant gel or powder, sponge particles, or any other absorptive material or structure). For example, an absorptive material and/or structure could be applied to the tendon(s) 130 of interest, such that aspiration of the excess tendon fluid through drainage structure(s) 150 would involve drawing the hydrated material/structure through drainage structure(s) 150. In such cases, drainage structure 150 may or may not be inserted directly into sheath 130-S of tendon 130 (e.g., a desiccant placed around tendon 130 could draw fluid out of tendon 130, at which point the hydrated desiccant could be aspirated without having to puncture sheath 130-S).

Although this aspiration procedure can be performed as an open or mini open procedure via one or more incisions in the skin of the patient, the use of drainage structure 150 can eliminate the need for such incisions, thereby minimizing trauma to the patient and subsequent recovery time. Specifically, in various embodiments, drainage structure 150 can itself be used to puncture the skin of hand 100 (and any intervening structures) to access and aspirate tendon(s) 130. For example, in some embodiments, drainage structure 150 can pierce both the skin and transverse carpal ligament 110 to reach tendon(s) 130. In such minimally invasive approaches, fluoroscopic, ultrasonic, endoscopic, or other visualization or navigation methods can optionally be used to ensure proper placement of drainage structure 150.

In various embodiments, the tendon aspiration procedure described with respect to FIG. 1C can be supplemented with local therapeutic substance delivery (before, after, and/or during aspiration). For example, an anti-inflammatory drug, antibiotic, corticosteroid, pain medication, analgesic, anesthetic, relaxant, enzyme, lubricant, anti-fibrotic agent, desiccant, growth factor, and/or other therapeutic agent could be delivered in conjunction with tendon aspiration to provide enhanced or broader symptom management. In various embodiments, the therapeutic substance can be a liquid, gel, or solid (including particulate compositions). In various other embodiments, the therapeutic substance may be short-acting or formulated in a device or delivery mechanism that enables extended release and/or controlled release.

In some embodiments, especially if tendon aspiration is performed in an open procedure, the therapeutic substance delivery can be via direct swabbing or placement of a therapeutic substance release carrier (e.g., a drug-infused sponge or implantable pump) in the region of carpal tunnel 105. However, in various other embodiments, therapeutic substance delivery can be via needles to match similarly minimally invasive tendon aspiration steps.

For example, in one embodiment, therapeutic substance delivery can be performed through the same drainage structure(s) 150 used to aspirate flexor tendon(s) 130. For example, as indicated in FIG. 1C, drainage structure 150 can be decoupled from flow controller 160 and coupled to an optional source 161 for therapeutic substance delivery. Drainage structure 150 can either be left in place or repositioned prior to therapeutic substance delivery.

In various other embodiments, separate aspiration and therapeutic substance delivery instruments can be used. For example, FIG. 1D shows a delivery structure 151 coupled to a source 161 for delivering a therapeutic substance to tendon(s) 130. Note that while source 161 is depicted as being remotely coupled to delivery structure 151 (e.g., via flexible tubing) for exemplary purposes, in various other embodiments, source 161 could be rigidly coupled to delivery structure 151 (e.g., a syringe fitted with delivery structure 151).

In some embodiments, structures 150 and 151 can be integrated in a single unit (e.g., a double-barreled or coaxial needle construction). In various other embodiments, structures 150 and 151 can be discrete elements. For example, separate needles would allow for aspiration and therapeutic substance delivery operations at different locations concurrently or in different sequences.

For example, in some embodiments, aspiration of flexor tendon 130 can be performed through drainage structure 150 and therapeutic substance delivery can be to transverse carpal ligament 110 via delivery structure 151, as shown in FIG. 1E. Such a procedure could involve, for example, the delivery of a relaxant and/or growth factor to ligament 110 to encourage lengthening, thereby providing an additional increase in carpal tunnel space.

In some other embodiments, aspiration of flexor tendon 130 can be performed through drainage structure 150 and therapeutic substance delivery can be to the general area around carpal tunnel 105, as shown in FIG. 1F. Such a procedure could involve, for example, the delivery of a corticosteroid (optionally with a local anesthetic) to reduce swelling in addition to the aspiration. Various other combinations and sequences of aspiration and therapeutic substance delivery will be readily apparent.

Note that while the above-described methodology would typically be performed using needles for aspiration and/or drainage, in various other embodiments, drainage structure(s) 150 and/or delivery structure(s) 151 could be any structure providing aspiration or delivery functionality, respectively. For example, FIG. 1G shows an embodiment in which drainage structure 150 is implemented as an implanted shunt, through which aspiration of flexor tendon 130 is controlled by flow controller 160, which could be a valve or pump. In such an embodiment, drainage structure 150 could lead to another part of the body (e.g., the abdominal cavity) that could easily accept and resorb the excess fluid removed from flexor tendon 130. Various other embodiments of drainage structure 150 will be readily apparent.

In any event, the reduction in the swelling of flexor tendon(s) 130 can result in significantly less squeezing of median nerve 120, as depicted in the exemplary post-procedure cross section A-A of hand 100 shown in FIG. 1H. Because aspirated flexor tendons 130 take up less space within carpal tunnel 105, median nerve 120 is no longer compressed against transverse carpal ligament 110, and the CTS-related discomfort associated with such compression can be reduced or eliminated. This decompression is provided without damaging transverse carpal ligament 110, and so does not result in the problematic side effects associated with conventional carpal tunnel release surgery.

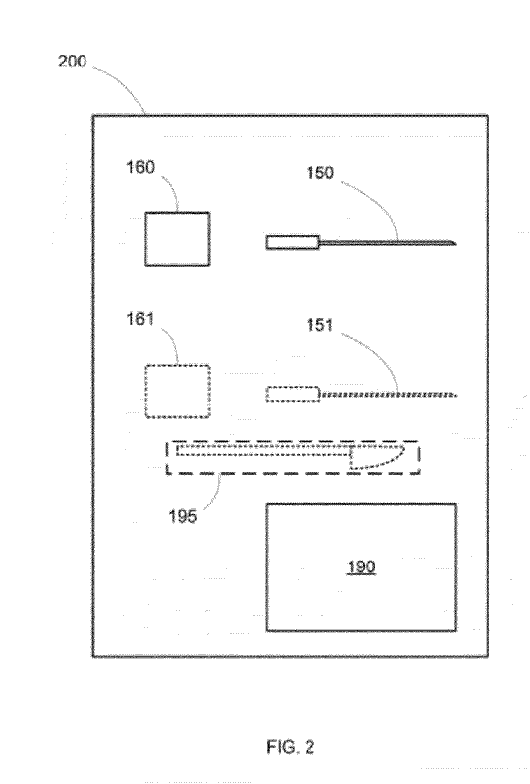

FIG. 2 shows an exemplary surgical kit 200 that includes a drainage structure 150 (e.g., a needle or shunt) and a flow controller 160 (e.g., a syringe, pump, or valve), such as described above with respect to FIGS. 1C-1F. Note that while a single drainage structure 150 and flow controller 160 are depicted for clarity, kit 200 can include any number of drainage structures 150 and/or flow controllers 160.

Kit 200 further includes instructions 190 that explain one or more uses of drainage structure 150 and flow controller 160, such as described in FIGS. 1C-1F. For example, in some embodiments, instructions 190 can describe coupling flow controller 160 to drainage structure 150, optionally piercing the sheath (e.g., sheath 130-S) of a tendon (e.g., flexor tendon 130), and aspirating the tendon by withdrawing fluid from the tendon through drainage structure 150. In various embodiments, instructions 190 can specify a maximum quantity of fluid to be aspirated from the tendon. In various other embodiments, instructions 190 can specify visual cues indicating proper placement of drainage structure 150 at the tendon and/or appropriate swelling reduction of the tendon (either via direct visualization in an open procedure or via indirect visualization via endoscopic, ultrasonic, fluoroscopic, or navigation equipment).

In some embodiments, kit 200 can further include one or more delivery structures 151 (e.g., needles) and/or sources 161 (e.g., mixers, syringes, and/or vials/ampoules of drugs) for therapeutic substance delivery to the affected area. In such embodiments, instructions 190 can further specify the usage of delivery structure(s) 151 and/or source(s) 161 for therapeutic substance delivery in conjunction with the aspiration provided by drainage structure 150 and flow controller 160, such as described with respect to FIGS. 1C-1F.

In various other embodiments, kit 200 can include optional additional instruments 195, such as cutting tools (e.g., a curette or scalpel), retractors, and/or mixers, among others. For example, additional instruments 195 could include a scalpel for creating an incision for allowing open access to the flexor tendons for aspiration.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

1. A method for treating carpal tunnel syndrome in a patient, the method comprising:
placing a drainage structure in or adjacent to a carpal tunnel in the patient; and
aspirating a first flexor tendon through the drainage structure to reduce swelling of the first flexor tendon within the carpal tunnel.

2. The method of claim 1, wherein the drainage structure comprises a first needle coupled to a syringe, and
wherein aspirating the first flexor tendon comprises aspirating at or near the first flexor tendon through the first needle into the syringe.

3. The method of claim 2, wherein the patient comprises skin covering the carpal tunnel, and
wherein placing the drainage structure in or adjacent to the carpal tunnel comprises piercing the skin with the first needle.

4. The method of claim 3, wherein the carpal tunnel comprises a transverse carpal ligament, and
wherein placing the drainage structure in or adjacent to the carpal tunnel further comprises piercing the transverse carpal ligament with the needle.

5. The method of claim 1, wherein the drainage structure comprises a shunt.

6. The method of claim 1, further comprising:
aspirating a second flexor tendon through the drainage structure to reduce swelling of the second flexor tendon within the carpal tunnel.

7. The method of claim 1, further comprising:
placing a second drainage structure in or adjacent to the carpal tunnel; and
aspirating at or near a second flexor tendon through the second drainage structure to reduce swelling of the second flexor tendon within the carpal tunnel.

8. The method of claim 1, further comprising delivering a therapeutic substance adjacent to or within the carpal tunnel.

9. The method of claim 8, wherein the therapeutic substance comprises at least one of an anti-inflammatory, an antibiotic, a corticosteroid, an analgesic, an anesthetic, an enzyme, a lubricant, an anti-fibrotic agent, a desiccant, a growth factor, and a relaxant.

10. The method of claim 8, wherein delivering the therapeutic substance comprises conveying the therapeutic substance through the drainage structure.

11. The method of claim 8, wherein delivering the therapeutic substance comprises:
placing a delivery structure in or adjacent to the carpal tunnel; and
conveying the therapeutic substance through the delivery structure.

12. The method of claim 8, wherein the carpal tunnel comprises a transverse carpal ligament, and
wherein delivering the therapeutic substance comprises injecting the therapeutic substance into the transverse carpal ligament.

13. A kit comprising:
a first drainage structure; and
a set of instructions describing the use of the first drainage structure to aspirate a first flexor tendon.

14. The kit of claim 13, further comprising a flow controller, wherein the set of instructions further describes coupling the first drainage structure to the flow controller to draw fluid from the first flexor tendon through the first drainage structure.

15. The kit of claim 13, wherein the set of instructions further describes using the first drainage structure to aspirate a second flexor tendon.

16. The kit of claim 13, further comprising a second drainage structure, wherein the set of instructions further describes using of the second drainage structure to aspirate a second flexor tendon.

17. The kit of claim 13, wherein the first drainage structure comprises a needle.

18. The kit of claim 13, wherein the first drainage structure comprises a shunt.

19. The kit of claim 13, further comprising a source, wherein the set of instructions further describes using the first drainage structure to deliver a therapeutic substance from the source.

20. The kit of claim 13, further comprising an access instrument, wherein the first set of instructions further describes using the access instrument to create an access opening for the first drainage structure.
